(19) United States
(12) Patent Application Publication
(10) Pub. No.: US 2018/0168556 A1
(43) Pub. Date: Jun. 21, 2018

(54) Capillary Slit Urine Sampling System

(71) Applicants: David R. Hall, Provo, UT (US); Dan Allen, Springville, UT (US); Jared Reynolds, Pleasant Grove, UT (US); Joshua Larsen, Spanish Fork, UT (US); Terrece Pearman, Draper, UT (US)

(72) Inventors: David R. Hall, Provo, UT (US); Dan Allen, Springville, UT (US); Jared Reynolds, Pleasant Grove, UT (US); Joshua Larsen, Spanish Fork, UT (US); Terrece Pearman, Draper, UT (US)

(21) Appl. No.: 15/383,187

(22) Filed: Dec. 19, 2016

(51) Int. Cl. A61B 10/00; E03D 11/02; G01J 3/02

(52) U.S. Cl. CPC A61B 10/007; G01J 3/0205; E03D 11/02

(57) ABSTRACT

We disclose a device and method for collecting a urine sample as a user urinates into a toilet. The device further conducts analytical measurements which may include spectral, colorimetric, and chemical assays. The user need only urinate normally into the toilet. A urine collection trap, which includes a vertical slit, may capture a urine sample which has a volume in the microliter range. Pumps may divert the urine from the vertical slit into a conduit that is connected to spectral analysis devices and other devices for analyzing the urine sample. The disclosed device is both convenient and prevents unsanitary urine spills.

### BACKGROUND

### Field of the Invention

This invention relates to devices for collecting and analyzing urine and methods of use thereof.

### Background of the Invention

Collecting a urine sample for analysis is often inconvenient. Urine collection often involves urinating into a receptacle and may result in unsanitary urine spills and drips. Some types of urinalysis require a user to urinate on a test strip or a specific part of a device. Controlling the urine stream to contact only a test strip or part of a device is often difficult and results in urine splashing onto unwanted areas. A device and method of collecting a urine sample that is easy and without risk of urine spills or drips is needed.

In addition, many types of urinalysis assays are not adapted for use outside the clinic setting. Consequently, a device and method of performing complex analysis of urine samples in the home or elsewhere outside of the clinic setting is needed.

### SUMMARY

We disclose a novel system for collecting and analyzing urine samples. The urine sampling system may be located within a toilet bowl. The urine sampling system may include an elongated, elevated mound that may be flush with the wall of the toilet bowl. A vertical slit may bisect the mound with the slit running substantially parallel with the longitudinal axis of the mound. A user urinates normally into the toilet bowl and the urine stream flows over the mound. The mound acts as a dam to channel urine into the vertical slit. The vertical slit captures a urine sample by capillary action.

The urine sampling system may include a conduit that is in fluid connection with the vertical slit and may include a pump that, when actuated, moves the urine sample from the vertical slit into the conduit through a sample port. The conduit may be in fluid connection with one or more analytical devices which may conduct analytical assays on the urine sample. These assays may include, but are not limited to, chemical, spectral, and colorimetric assays.

The urine sampling system may include a junction along the conduit that includes a spectral analysis cell. The spectral analysis cell may include two substantially parallel light-transmitting plates. The urine may move from the conduit into the space between the light-transmitting plates. A light source may emit light through a filter which then allows light of one or more defined wavelengths to pass through the filter and through the urine sample in the spectral analysis cell. A spectrometer may then perform a spectral measurement on the light transmitted through the spectral analysis cell. In some embodiments, the urine sampling system may store the urine sample after analysis for additional testing.

The urine may continue through the conduit after passing through the junction to either exit the urine sampling system or be transferred into additional medical devices for further analysis. In some embodiments, the urine exits the conduit through a port that is different than the sample port and in some embodiments the pump reverses the direction of urine travel sending it out through the sample port.

Upon exiting the urine sampling system, the urine may be dispensed into the toilet water and flushed. The urine sampling system may draw rinse water through the system between uses. Urine flows around the mound, rather than pooling, so that there is little or no residual urine remaining on the mound. Rinse water from the lip of the toilet bowl that may be dispensed upon flushing the toilet or water from a sprayer may rinse the mound between uses.

The disclosed urine sampling system collects a urine sample while the user simply urinates into a toilet bowl thus providing a convenient and sanitary method to collect urine for analysis. The urine sampling system may then perform one or more analysis on the sample which may be used to assess the user's health status or diagnose illness. The analysis may be done in the home or elsewhere outside of a clinical setting. The disclosed urine sampling system provides convenient health services without the need to visit a clinic.

### BRIEF DESCRIPTION OF THE DRAWINGS

FIG. 1 is a perspective drawing of a toilet including an embodiment of the disclosed urine sampling system.

FIG. 2 is a close-up view of an embodiment of the disclosed urine sampling system

FIG. 3 is an aerial view a toilet including an embodiment of the disclosed urine sampling system.

FIG. 4 is a cross-sectional schematic view of a toilet including an embodiment of the disclosed urine sampling system and two urinalysis assay devices.

FIG. 5 is a schematic drawing of an embodiment of the disclosed urine sampling system which includes a peristaltic pump.

FIG. 6 is a schematic drawing of an embodiment of the disclosed urine sampling system which includes a syringe pump.

FIG. 7A is a schematic drawing illustrating a side view of an embodiment of a spectral analysis cell that may be connected to the disclosed urine sampling system.

FIG. 7B is a schematic drawing illustrating a front view of the spectral analysis cell of FIG. 7A.

FIG. 8 is a schematic drawing of the spectral analysis cell of FIGS. 7A and 7B and a spectrometer which may be connected to the disclosed urine sampling system.

FIG. 9 is a schematic drawing of an embodiment of the disclosed urine sampling system in which a single orifice functions as the sample port and the output port.

FIG. 10 is a flow chart illustrating an embodiment of a process through which an embodiment of the disclosed urine sampling system may function.

### DETAILED DESCRIPTION

### Definitions

User, as used herein, means a human or animal that deposits bodily waste into an embodiment of the toilet disclosed herein.

Toilet, as used herein, means a device that may be used to collect one or more biological waste products of a user.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

Disclosed herein is a urine sampling system which comprises a device which captures a urine sample as a user urinates into a toilet. Upon capturing the urine sample, the device may transfer the urine sample into one or more analytical devices which may conduct one or more chemical, colorimetric, or spectral analysis on the urine sample. The measurements collected from the analytical devices may be used to provide an assessment of the user's health or provide a diagnosis. In some embodiments, the urine sampling system then captures a sample of water to rinse the system between uses.

Parts of the disclosed urine sampling system may be positioned within the toilet bowl of a toilet. In some embodiments, the urine sampling system includes a structure that resembles an elongated mound that may be an elevated region of the wall of the toilet bowl. The mound may be formed from two separate sections separated by a vertical slit. The mound may include a longitudinal and transverse axis, the longitudinal axis being greater in length than the transverse axis. The vertical slit may run substantially parallel to the transverse axis and may approximately bisect the mound between the two sections. In some embodiments, the longitudinal axis of the mound is parallel to an axis of the toilet bowl that runs left to right across the toilet bowl from the perspective of a user sitting on the toilet seat.

The mound may be positioned above the standing water level set by the toilet's P-trap. Furthermore, the mound may be located near the front of the toilet bowl. As used herein, the front of the toilet bowl is the side of the toilet bowl where a user may stand as he or she approaches the toilet bowl. The front is opposite a rear side of the toilet bowl which is near the opening which leads to the P-trap in a traditional toilet. Consequently, the user's urine stream flows onto the mound whether the user is standing in front of the toilet or sitting on the toilet seat.

The mound functions much like a dam by temporarily detaining urine as it flows over the mound and channeling a urine sample into the vertical slit. In some embodiments, the vertical slit collects approximately between 10 μl of urine to approximately 50 μl of urine. In some embodiments, the vertical slit collects approximately 25 μl of urine. The width of the vertical slit may be small enough that capillary forces hold urine in the vertical slit even after urination has stopped. However, the urine does not pool on the mound so rinse water from the lip of the bowl or a sprayer may rinse the residual urine from the mound and may provide rinse water that enters the urine sampling system through the vertical slit as discussed in more detail below. Consequently, the urine sampling system may be rinsed when the user flushes the toilet.

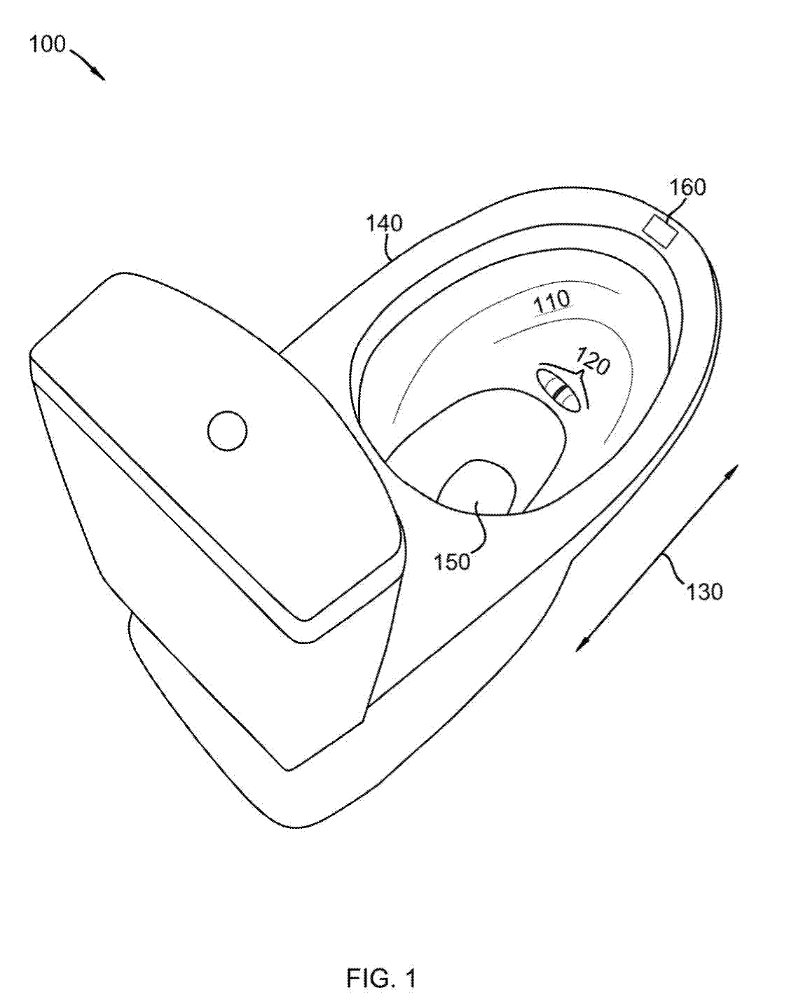

In some embodiments, the urine sampling system includes a vertical slit but does not include a mound. In these embodiments, the slit is positioned substantially flush with the wall of the toilet bowl. A user's urine stream may pass over the vertical slit. The vertical slit may pull a urine sample into the vertical by capillary action. The vertical slit may be substantially perpendicular to a front-to rear axis of the toilet bowl, substantially parallel to the front-to-rear direction axis of the toilet bowl, or positioned at an angle between parallel and perpendicular to the front-to-rear direction axis of the toilet bowl. “Front-to rear direction axis” as used herein, is illustrated as front-to-rear direction axis 130 shown in FIG. 1. “Front” is the side of the toilet bowl that is nearest where a user approaching the toilet would stand and “rear” is the side along front-to-rear direction axis 130 that is nearest the toilet tank of a traditional toilet.

In some embodiments in which the vertical slit is positioned substantially flush with the wall of the toilet bowl, the toilet bowl may include a depression in a wall of the toilet bowl. The vertical slit may be located within the depression. Urine may flow down into the depression and into the vertical slit. The vertical slit may then pull a urine sample into the vertical by capillary action.

In some embodiments in which the vertical slit is positioned substantially flush with the wall of the toilet bowl, the wall of the toilet bowl may include an elongated channel. The elongated channel may trap urine and direct the urine toward the vertical slit.

In some embodiments, the urine sampling system includes one or more sensors which detect the presence of urine. The sensors may include a temperature sensor, an optical sensor, or both. In some embodiments, a level sensor or flow meter is present in the toilet bowl or P-trap which detects when volume has been added to the toilet bowl. A gas sensor may be used in conjunction with the level sensor or flow meter to detect volatile organic compounds emitted from feces. Thus, the sensors may distinguish between defecation and urination events.

Some embodiments include sensors which may detect the presence of a user on or near the toilet. These sensors may include optical proximity sensors and weight sensors. In addition, weigh sensors may detect that the user is urinating by detecting a loss of weight after an initial body weight measurement. The weight sensor may also be combined with a gas sensor to differentiate weight lost due to urination versus defecation.

A sampling port may be positioned either at the bottom or a side of the vertical slit. The sample port may connect the vertical slit with a conduit. The conduit may resemble a section of tubing or a pipe. Some embodiments include an inlet valve between the sample port and the conduit which regulates fluid entry into the system and fluid exit from the system. The inlet valve may be connected to a controller which may actuate the valve to open and close the valve.

In some embodiments, the conduit is connected to a T-junction which includes three ports. The sample port may comprise a first port of the T-junction. A second port of the T-junction may be connected to a pump which, when actuated, may create force which pulls urine from the vertical slit, through the sample port, and into the conduit. A hose or similar tubing may connect the pump to the conduit. A third port of the T-junction may be an output port through which urine exits the urine sampling system after analysis. Some embodiments include an output valve which may open to allow fluid to exit the urine sampling system after urinalysis but which may close during and prior to urine analysis.

The pump that is present in embodiments that include a T-junction may be a syringe pump. In embodiments which include check valves, the action of the syringe pump may open and close the valves without the need for a signal from the controller.

The syringe pump may draw a urine sample into the conduit from the vertical slit. The pump may draw the entire capillary volume, after which the pump will draw air. This creates a narrow bubble of urine with an inner diameter of approximately between 1/32 inch and approximately 1/16 inch. This allows for a smaller syringe pump volume which may make dispensing small volumes easier. In embodiments in which the output valve is closed at this point in the process, the bubble may be drawing through the second port of the T-junction towards the syringe pump. We point out that some embodiments do not create a narrow bubble of urine. Embodiments which create a narrow bubble of urine simply do so to enable a smaller syringe pump volume.

Alternatively, other embodiments may not include a T-junction. Rather, some embodiments may include a linear fluid path which may include a conduit and a peristaltic pump which pushes fluid through the conduit. In embodiments that include a linear fluid path with no T-junction, fluid may enter the sample port and travel through the conduit then exit through the outlet port. In these embodiments, the peristaltic pump pushes fluid through the conduit in a single direction.

In other embodiments, a single port functions as both the sample port and output port. The urine sample may travel from the vertical slit through the single port and into the conduit where it is analyzed. A peristaltic pump may function in a first direction to force the urine from the vertical slit toward the conduit. After analysis is complete, the peristatic pump may function in a second direction to force urine from the conduit and back through the single port and out through the vertical slit.

In embodiments that include a T-junction, a syringe pump may pull fluid into the conduit through the single port when the plunger is withdrawn from the barrel of the syringe pump. After analysis, the plunger may re-enter the barrel of the syringe pump creating a force which pushes the fluid back through the conduit in the opposite direction the urine traveled during entry, through the single port, and out through the vertical slit.

In embodiments that include a single port that functions as both a sample port and output port, valves to regulate fluid entry into and exit from the conduit are optional. In embodiments that include a single sample/outlet port, a valve may optionally be included to prevent entry of foreign substances into the system during urinalysis. The valve may be a solenoid driven pinch valve or other actuated valve.

Upon entering the conduit, the urine may travel to a junction which may be positioned along the conduit. The junction may include a spectral analysis cell which may house urine while the urine sampling system conducts optical measurements. The optical measurements may detect one or more of color, light transmission, and particle size. The measurement may be performed by microscopy, laser light scattering, turbidity measurements, spectroscopy, or other techniques known in the art.

In some embodiments, the spectral analysis cell may include two light-transmitting plates which may be positioned substantially parallel to each other. A width of a space between the two light-transmitting plates may be small enough that the urine may spread substantially evenly along the faces of the plates by capillary force. The width of the space between the two light-transmitting plates may be adjustable so as to adjust the distance of the light transmission path through the urine sample. In some embodiments, a variable diaphragm, which may be a liquid lens, may be used to control the length of the light transmission path thereby controlling the spectral measurements. Some embodiments include a compliant seal, which may be an O-ring, between the two light-transmitting plates. The compliant seal may contain the urine sample keeping it within a light transmission section through which light emitted from a light source may travel during spectral analysis. In some embodiments, an entrance port transverses one of the light-transmitting plates. The urine sample may travel from the conduit into the spectral analysis cell through the entrance port. The urine may exit the spectral analysis cell through an exit port that may transverse the second light-transmitting plate. Both the entrance port and the exit port may be in fluid connection with the conduit through sections of tubing or pipe.

Spectral analysis may also be conducted through tubing that comprises one or more sections of the conduit. The cross-sectional diameter of the tubing may vary along the length of the tubing. Spectral measurements may be collected from sections of tubing with different diameters to collect measurements from regions of tubing that have different levels of absorption.

Embodiments which include a microfluidic system for spectral analysis that is located below the mound and vertical slit may have an advantage over conducting the spectral analysis in the vertical slit. These may include additional protection from ambient light. In addition, it may be easier to regulate the temperature within the microfluidic system below the vertical slit. This is at least because urine in the vertical slit may evaporate, lose heat, and may absorb heat from the toilet bowl.

In some embodiments, the third port of a T-junction or yet another port may connect the conduit with a reservoir housing a reagent which may be added to the urine sample within the spectral analysis cell to conduct a chemical or colorimetric assay.

In addition to spectral analysis, the conduit may be connected to other devices which may conduct urinalysis assays. In some embodiments, the urine is expelled through an output port and into a reagent, or test strip for further analysis. In some embodiments, the urine exits the output port and is dispensed into a multi-well plate for further chemical analysis. The output port may dispense the urine through a volume controlled system known in the art which may include an automated micropipette.

In some embodiments, the urine may be dispensed into a collector for further examination or to maintain as evidence. This step may be performed when an analytical test indicates a potential health concern while non-suspect samples may be ejected out through the vertical slit or a separate output port.

Referring now to the drawings, FIG. 1 illustrates toilet 100 which includes an embodiment of the disclosed urine sampling system. Toilet 100 includes rim 140 which is connected to proximity sensor 160. In some embodiments, proximity sensor 160, or embodiments thereof, detect the presence of a user and send a signal to a controller which then prepares the urine sampling system to receive a urine sample.

Toilet 100 further includes toilet bowl 110 and orifice 150 which leads to the P-trap within toilet 100. Urine collection trap 120 is shown within toilet bowl 110. Urine collection trap 120 is positioned above the toilet water line and nearer the front of rim 140 than the rear of rim 140. In this embodiment, urine collection trap 120 is between the front of rim 140 and orifice 150. Arrow 130, shown in FIG. 1, illustrates the front-to-rear direction axis of toilet bowl 110. “Front,” as used herein, is the side along front-to-rear direction axis 130 that is nearest where a user approaching the toilet would stand and “rear” is the side along front-to-rear direction axis 130 that is nearest the toilet tank of a traditional toilet. The toilet bowl may also comprise a width axis which is perpendicular to the front-to-rear axis.

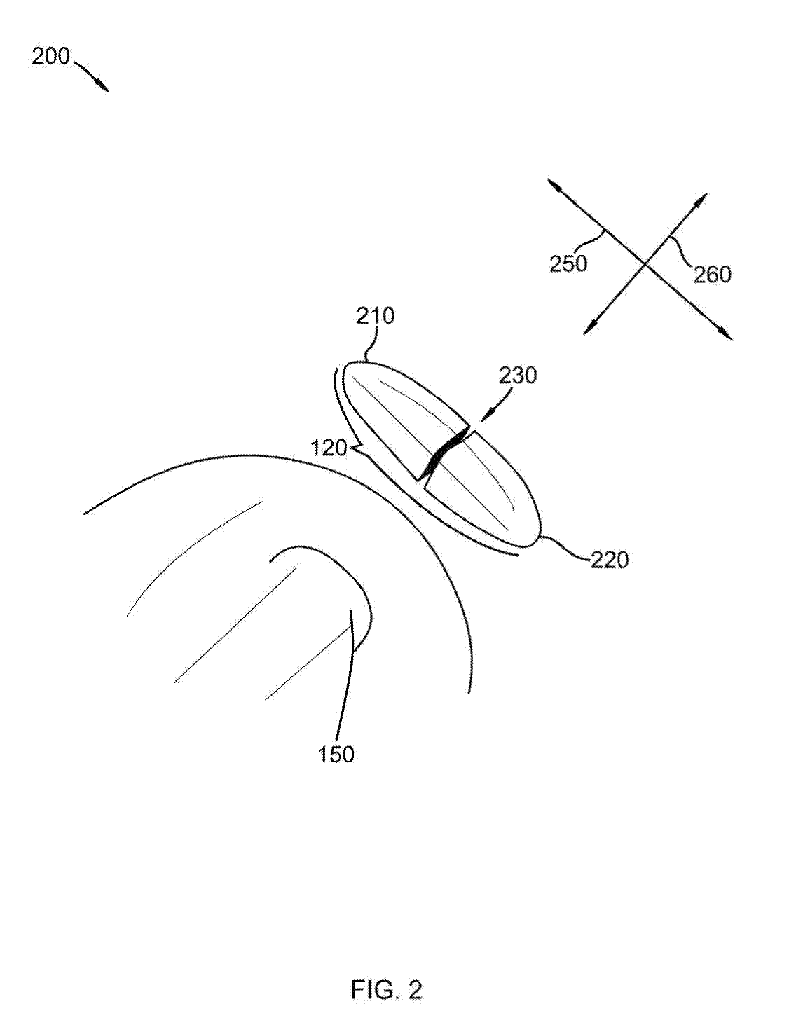

FIG. 2 is a close-up view of urine sampling system 200 which includes urine collection trap 120. Arrow 250 illustrates longitudinal axis of urine collection trap 120 and arrow 260 illustrates a transverse axis of urine collection trap 120. As described above with regard to FIG. 1, urine collection trap 120 is shown within toilet bowl 110 above the toilet water. Urine collection trap 120 resembles a mound which includes first section 210 and second section 220. The mound is elongated with the longer axis substantially parallel with longitudinal axis 250 and the shorter axis substantially parallel with transverse axis 260. An opening between first section 210 and second section 220 defines an orifice referred to herein as vertical slit 230. The orifice of vertical slit 230 transects the mound and is substantially parallel to front-to-rear direction axis 130 of the toilet bowl and to transverse axis 260 of urine collection trap 120. Vertical slit 230 is sufficiently narrow such that it captures urine and diverts it down into the orifice of vertical slit 230 by capillary action.

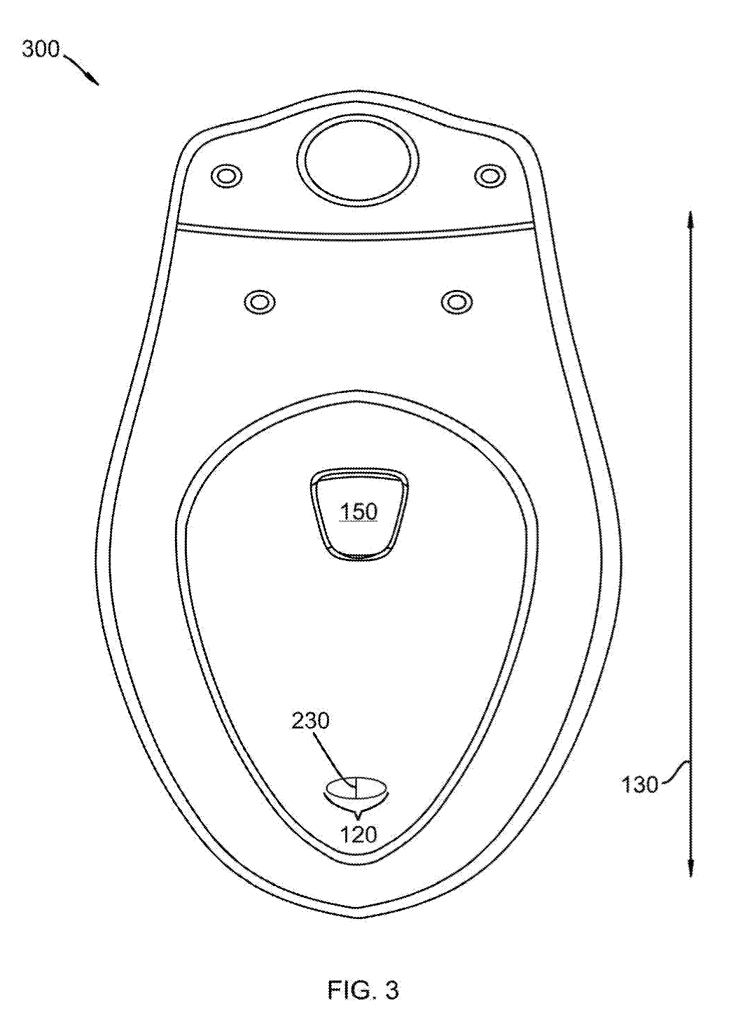

FIG. 3 is an aerial view of toilet 300 which includes an embodiment of the urine sampling system. Urine collection trap 120 is again illustrated nearer the front of the toilet bowl than orifice 150 and near the front of the toilet rim, with as defined by front-to-rear direction axis 130. In this embodiment, vertical slit 230 is substantially parallel to front-to-rear direction axis 130.

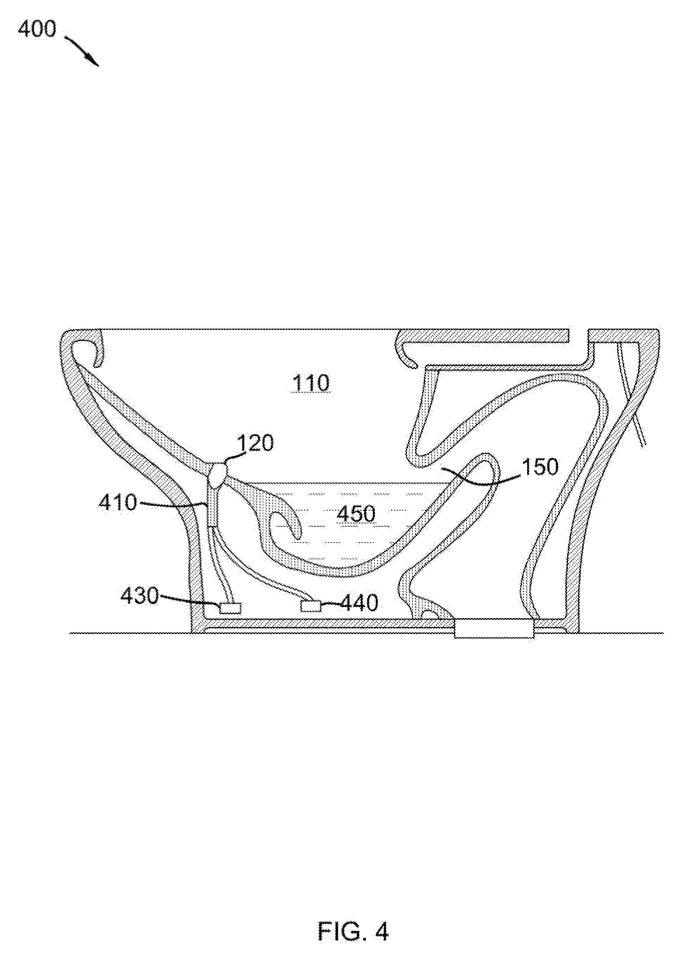

FIG. 4 is a cross-sectional view of toilet 400 which includes an embodiment of the disclosed urine sampling system. Toilet water 450 is shown within toilet bowl 110. Urine collection trap 120 is positioned within toilet bowl 110 above toilet water 450. Urine collection trap 120 is connected to conduit 410 which, in this embodiment, is located below toilet bowl 110. Urine collected by urine collection trap 120 is transferred through the vertical slit and into conduit 410. Conduit 410 is further connected to chemical analysis assay devices 430 and 440. Each of chemical analysis assay devices 430 and 440 may conduct a urine analysis assay on the urine sample.

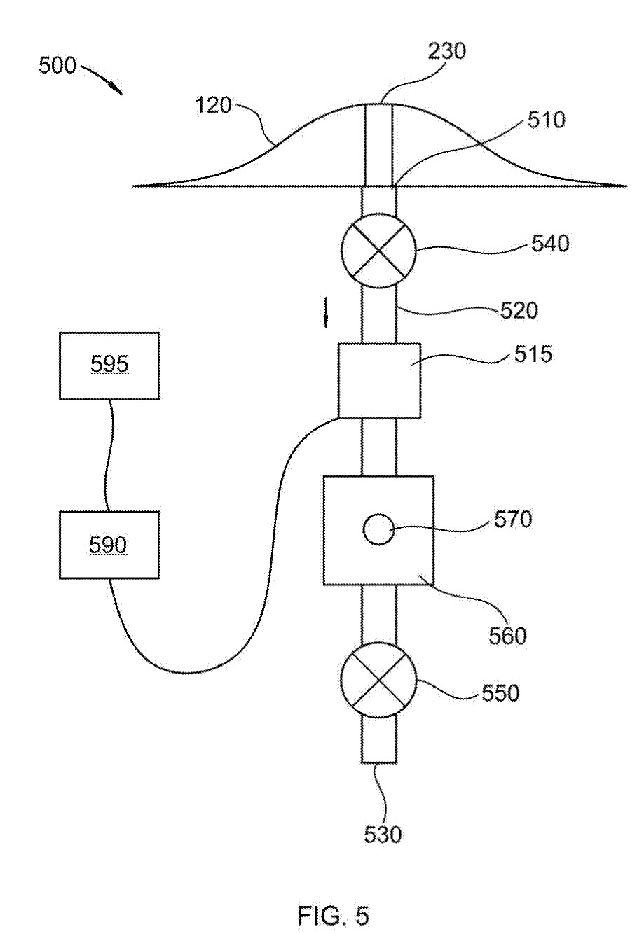

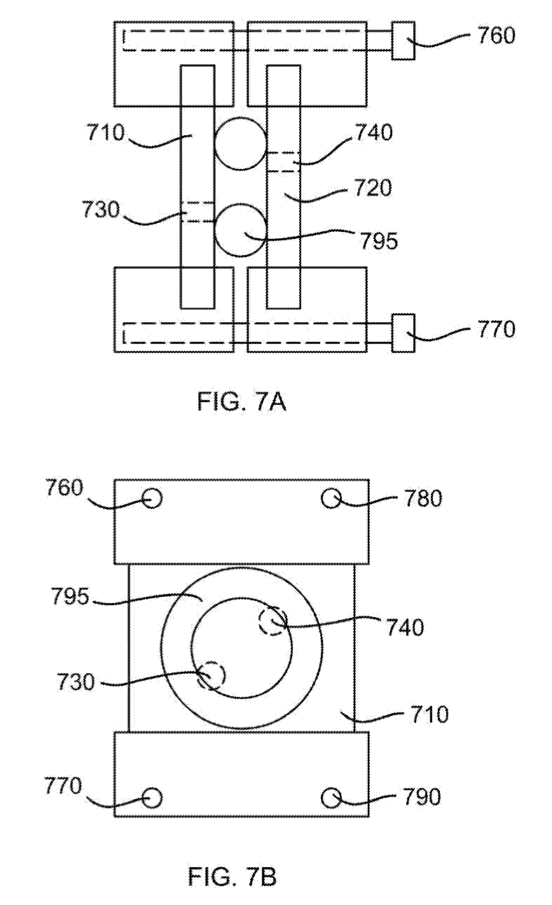

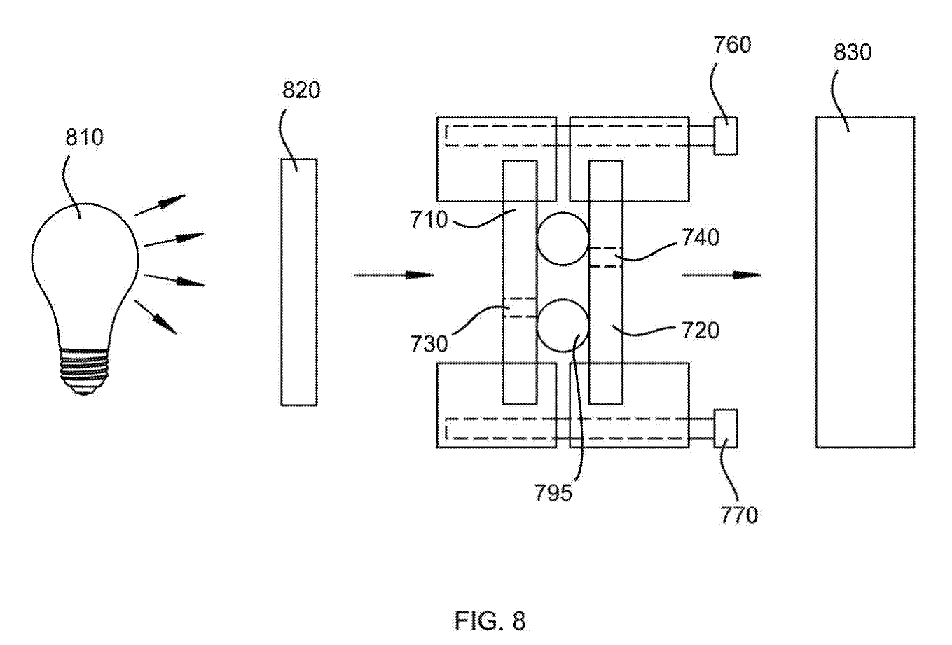

FIG. 5 is a schematic cross-sectional view of urine sampling system 500, which is an embodiment of the disclosed urine sampling system. Urine collection trap 120 is shown as a cross section of a mound with vertical slit 230 bisecting it. The mound configuration of urine collection trap 120 acts as a dam to trap urine as it flows into the toilet bowl. Urine pools against the mound as a dam inhibits water flow. The mound detains the urine long enough for some of the urine to enter vertical slit 230 but the remaining urine flows around the mound and into the toilet bowl so that there is no standing urine on urine collection trap 120. Capillary action pulls the urine sample from the upper opening of vertical slit 230 down into vertical slit 230. Conduit 520 is in fluid connection with vertical slit 230 through sample port 510. Urine sampling system 500 includes inlet valve 540 and output valve 540. Urine sensor 595 may detect the presence of urine and send a signal to controller 590. Controller 590 may then send a signal which causes inlet valve 540 to open and output valve 550 to close. Controller 590 may also send a signal to actuate peristaltic pump 515 which draws the urine sample from vertical slit 230 through sample port 510 and into conduit 520. The downward-pointing arrow indicates the direction of urine travel. As the urine travels through conduit 520, the urine enters a junction. In urine sampling system 500, the junction includes spectral analysis cell 560. An embodiment of a spectral analysis cell is shown in more detail in FIGS. 7A, 7B, and 8. Spectral analysis cell 560 includes light transmission section 570 through which filtered light may pass during spectral analysis. After spectral analysis is complete, controller 590 sends a signal to open output valve 540 and to actuate peristaltic pump 515. The urine sample moves out of spectral analysis cell 560, continues through conduit 520, and out of urine sampling system 500 through output port 530. In some embodiments, the process is repeated with rinse water between uses. In some embodiments, the rinse water may be toilet water.

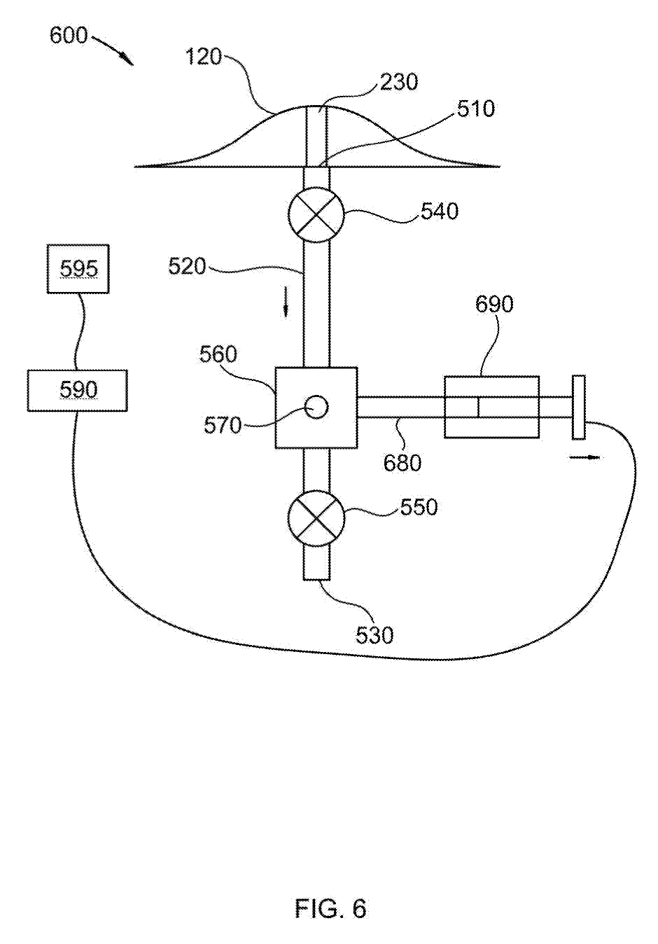

FIG. 6 is a schematic cross-sectional view of urine sampling system 600, which is another embodiment of the disclosed urine sampling system. Urine sampling system 600 is structurally and functionally similar to urine sampling system 500 except that peristaltic pump 515 of urine sampling system 500 has been replaced with syringe pump 690. A signal from controller 590 causes syringe pump 690 to actuate and the plunger of syringe pump 690 pulls away from the syringe barrel in the direction shown by the horizontal arrow in FIG. 6. Similar to peristaltic pump 515, syringe pump 690 pulls the urine sample from vertical slit 230 through sample port 510 and into conduit 520 in the direction shown by the downward-pointing arrow.

FIG. 7A is a cross sectional side view of an embodiment of a spectral analysis cell that may house a urine sample during spectral analysis and which may be included in the disclosed urine sampling system. The spectral analysis cell of FIG. 7A includes two light-transmitting plates 710 and 720 which are substantially parallel to each other. Entrance port 730 transverses light transmitting plate 710 and exit port 740 transverses the light-transmitting plate 740. Both entrance port 730 and exit port 740 are in fluid communication with the conduit of the urine sampling system. Urine may enter the spectral analysis cell from the conduit through entrance port 730 then exit the spectral analysis cell through exit port 740 which leads back into the conduit. Entrance port 730 and exit port 740 may connect to the conduit at substantially the same or different positions along the length of the conduit. The space between light-transmitting plates 710 and 720 defines a urine analysis reservoir which holds a urine sample while the urine sample undergoes spectral analysis. The width of the space between light-transmitting plates 710 and 720 may be adjustable to achieve optimal spectral measurements. In the embodiment of FIG. 7A, screws 760 and 770 may be turned to move light-transmitting plates 710 and 720 closer together or further from each other. This action adjusts the light path that a light source travels through during spectral analysis. In some embodiments, compliant seal 795 is positioned between light-transmitting plates 710 and 720. In some embodiments, compliant seal 795 is an O-ring seal.

FIG. 7B is a front view of the spectral analysis cell of FIG. 7B. In addition to screws 760 and 770 shown in FIG. 7A, screws 780 and 790 are visible in the view shown in FIG. 7B. Other embodiments may include more or fewer screws than shown in the embodiments of FIGS. 7A and 7B. In other embodiments, connectors known in the art, other than screws may be used to connect and move the light-transmitting plates.

FIG. 7B shows a front view of compliant seal 795 which, in this embodiment, is an O-ring seal. Alternatively, FIG. 7A illustrates a cross-section of the O-ring which is illustrated as two circles in FIG. 7A. Compliant seal 795 seals a small volume of urine in a light transmission section of the urine analysis reservoir through which a light source may pass during spectral analysis. Because compliant seal 795 is compliant, it may condense and expand as screws 760, 770, 780, and 790 adjust the distance between light-transmitting plates 710 and 720.

FIG. 8 is a schematic drawing of the spectral analysis cell of FIGS. 7A and 7B as it may be used to conduct a spectral measurement on a urine sample. The spectral analysis cell is shown as a cross-sectional side view as first illustrated by FIG. 7A. Light source 810 emits light of multiple wavelengths which pass through filter 820. A select range of wavelengths or a specific wavelength passes through filter 820 and then sequentially through light-transmitting plate 710, a urine sample within the spectral analysis cell, then light-transmitting plate 720. The light that is transmitted through light-transmitting plate 720 is then measured by spectrometer 830.

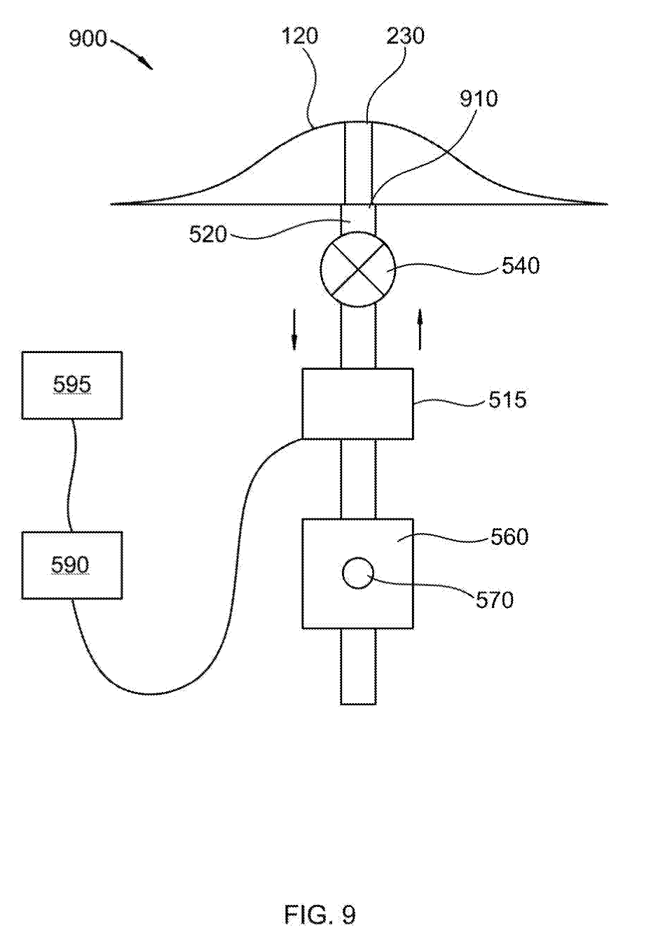

FIG. 9 is a schematic cross-sectional view of urine sampling system 900, which is yet another embodiment of the disclosed urine sampling system. Urine sampling system 900 is similar to urine sampling system 500 of FIG. 5. However, urine sampling system 900 includes sample/output port 910. This embodiment includes a single port that functions both as a sample port and an output port. Urine sensor 595 may detect the presence of urine and send a signal to controller 590. Controller 590 may then send signals which cause inlet valve 540 to open and peristaltic pump 515 to actuate. Because there is no separate output valve in this embodiment, controller 590 does not send a signal to close a second valve as in the embodiments of FIGS. 5 and 6. Peristaltic pump 515 may function in a first direction which may pull urine from vertical slit 230, through sample/outlet port 910, and into conduit 520. In some embodiments, controller 590 may send a second signal to inlet valve 540 causing it to close. The urine may enter a junction which includes spectral analysis cell 560. After analysis is complete, controller 590 may send another signal to peristaltic pump 515 which causes peristaltic pump 515 to function in a second direction. In some embodiments, controller 590 may send a second signal to inlet valve 540 causing it to open. In response to peristaltic pump 515 functioning in the second direction, urine may move back up conduit 520 and out through sample/outlet port 910.

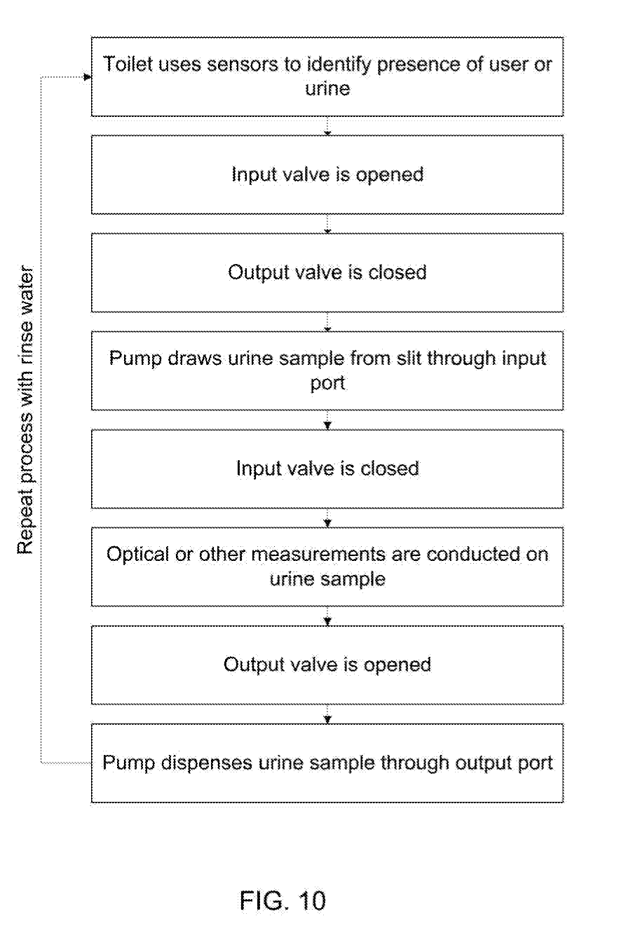

FIG. 10 is a flow chart illustrating a series of steps which the disclosed urine sampling system may conduct to collect and analyze a urine sample. The embodiment of FIG. 10 includes sensors that identify the presence of a user near the toilet or the presence of urine in the urine sampling system. Upon receiving the signal that a user or a urine sample is present, a controller sends a signal to the input valve on the conduit causing the input valve to open and a signal to the output valve on the conduit causing the output valve to close. Urine enters the vertical slit in the urine collection trap and is drawn into the urine collection trap through capillary action. A pump draws the urine sample from the vertical slit, through the input port and into the conduit. The sample is then subjected to one or more analysis assays which may include optical measurements and chemical analysis assays. The output valve is then opened and the urine sample exits the urine sampling system through the output port. In some embodiments, the urine sample is dispensed from the output port into the toilet water. In some embodiments, the urine sampling system is then rinsed by repeating the above-described steps with toilet water prior to the next user approaching the toilet.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A urine sampling system comprising:
a toilet, the toilet comprising:
a toilet bowl, the toilet bowl comprising a front-to-rear direction axis and a width axis;
a urine collection trap, wherein the urine collection trap is positioned on a wall of the toilet bowl, the urine collection trap comprising:
a vertical slit, wherein the vertical slit comprises a first orifice, and wherein the vertical slit captures urine by capillary action;
a conduit in fluid communication with the vertical slit;
a sample port, wherein the sample port is defined by a second orifice, and wherein the sample port defines a fluid connection between the vertical slit and the conduit;
a pump, wherein the pump is mechanically connected to the conduit, and wherein the pump moves urine through the conduit.

2. The urine sampling system of claim 1, wherein the vertical slit is substantially parallel to the front-to-rear direction axis of the toilet bowl.

3. The urine sampling system of claim 1, wherein the vertical slit is substantially perpendicular to the front-to-rear direction axis of the toilet bowl.

4. The urine sampling system of claim 1, wherein the vertical slit is located within a depression in the wall of the toilet bowl.

5. The urine sampling system of claim 1, further comprising a channel, wherein the channel comprises an elongated indentation in the wall of the toilet bowl, and wherein the channel leads to the vertical slit.

6. The urine sampling system of claim 1, wherein the urine collection trap further comprises an elongated mound, the elongated mound comprising:
a longitudinal axis, wherein the longitudinal axis is substantially parallel to the width axis of the toilet bowl; and
a transverse axis;
wherein the first orifice of the vertical slit is defined by a space between a first section and a second section of the mound, wherein the vertical slit transects the mound running in a direction that is substantially parallel to the front-to-rear direction axis of the toilet bowl and substantially parallel to the transverse axis of the mound.

7. The urine sampling system of claim 1, wherein the pump comprises one of the following: a peristaltic pump and a syringe pump.

8. The urine sampling system of claim 1, wherein the sampling port is in fluid connection with a bottom or a side of the vertical slit.

9. The urine sampling system of claim 1, wherein the conduit further comprises an output port; and wherein the output port defines a fluid connection between the conduit and the toilet bowl.

10. The urine sampling system of claim 9, further comprising an output valve, wherein the output valve is positioned between the conduit and the output port.

11. The urine sampling system of claim 9, wherein the output port deposits a urine sample into a device which conducts a chemical or colorimetric analysis assay.

12. The urine sampling system of claim 1, further comprising an inlet valve, wherein the inlet valve is positioned between the conduit and the sample port.

13. The urine sampling system of claim 1, wherein a urine sample both enters and exits the urine sampling system through the sample port.

14. The urine sampling system of claim 1, further comprising a junction, wherein the junction is in fluid communication with the conduit, wherein the junction comprises a first light-transmitting plate and a second light-transmitting plate, wherein the first and second light-transmitting plates are substantially parallel to each other, and wherein a space between the first and second light-transmitting plates defines a urine analysis reservoir.

15. The urine sampling system of claim 14, further comprising an entrance port and an exit port, wherein the entrance port transverses the first light-transmitting plate and the exit port transverses the second light-transmitting plates, and wherein the entrance port and exit port are in fluid communication with the conduit.

16. The urine sampling system of claim 14, wherein a width of a space between the first and second light-transmitting plate is adjustable.

17. The urine sampling system of claim 16, wherein one or more screws adjust the width of the space between the first and second light-transmitting plate.

18. The urine sampling system of claim 14, further comprising a compliant seal, wherein the compliant seal is positioned between the first and second light-transmitting plate.

19. The urine sampling system of claim 14, further comprising a spectrometer, wherein the spectrometer measures one or more spectral properties of a urine sample between the first and second light-transmitting plates.

20. The urine sampling system of claim 1, further comprising at least one user sensor, wherein the at least one user sensor comprises one or more of the following: a proximity sensor and a weight sensor.
